(19) United States
(12) Patent Application Publication
(10) Pub. No.: US 2023/0260115 A1
(43) Pub. Date: Aug. 17, 2023

(54) RECORDING MEDIUM, DYNAMIC ANALYSIS SYSTEM, AND DYNAMIC ANALYSIS DEVICE

(71) Applicant: KONICA MINOLTA, INC., Tokyo (JP)

(72) Inventor: Tsuyoshi ITO, Tokyo (JP)

(73) Assignee: KONICA MINOLTA, INC., Tokyo (JP)

(21) Appl. No.: 18/105,079

(22) Filed: Feb. 2, 2023

(30) Foreign Application Priority Data
Feb. 14, 2022 (JP) 2022-020236

(51) Int. Cl. G06T 7/00; G16H 30/20; G16H 50/20

(52) U.S. Cl. CPC G06T 7/0012; G16H 30/20; G16H 50/20; G06T 2207/10081; G06T 2207/10088; G06T 2207/10116; G06T 2207/10132

(57) ABSTRACT

A recording medium storing a computer-readable program for modifying a dynamic analysis algorithm that performs dynamic analysis to a dynamic image, the program causing a computer to perform: a process of receiving, from a first data collection device, a first data set that is anonymized and includes a first dynamic image obtained by dynamic imaging with radiation on a first subject and information obtained by a first test other than the dynamic imaging on the first subject; a process of receiving, from a second data collection device, a second data set that is anonymized and includes a second dynamic image obtained by dynamic imaging with radiation on a second subject and information obtained by a second test other than the dynamic imaging on the second subject; and a process of modifying the dynamic analysis algorithm based on the first data set and the second data set.

### REFERENCE TO RELATED APPLICATIONS

The entire disclosure of Japanese Patent Application No. 2022-020236, filed on Feb. 11, 2022, including description, claims, drawings and abstract is incorporated herein by reference in its entirety.

### TECHNICAL FIELD

The present invention relates to a recording medium, a dynamic analysis system and a dynamic analysis device.

### DESCRIPTION OF THE RELATED ART

Recently, diagnoses have been attempted using the dynamic images obtained from dynamic imaging of patients with radiation. Since dynamic imaging is a new technology, the number of pieces of data for normal and abnormal cases is limited, and few physicians have knowledge of the dynamic images and their analysis.

The dynamic image has the advantage of providing more types of information than still images in that the dynamic image allows the movement of each tissue to be seen, but it also increases the review time per image for physicians who must review a large number of images daily. Therefore, it is more important than still images to provide physicians with the results of image analysis from the perspective of properly understanding information. that can only be obtained with the dynamic image without increasing the review time per image. For example, the dynamic state diagnosis support information generation system, which analyzes the dynamic image consisting of multiple frame images, discriminates between normality and abnormality, and generates the diagnosis support information. (see JP 2013-1691400 A).

In diagnosis and clinical research using the dynamic image, there is also a desire to view the dynamic image while contrasting it with the information obtained by conventional diagnosis methods other than dynamic imaging (medical images taken with other modalities, test results, etc.).

SUMMARY OF THE INVENTION

However, the dynamic image provides a much greater amount of information than a still image, and since it is a moving image, it can be used not only for morphological diagnosis but also for functional diagnosis, providing wide range of analysis results. The information obtained by the analysis of dynamic images is diverse, and the types of analysis are also diverse and complex. Furthermore, as the amount of dynamic image data collected increases, the analysis targets (e.g., the analysis target disease, respiratory analysis, blood flow analysis, organ migration analysis, etc.) will increase in variety to improve the analysis accuracy, and the dynamic analysis algorithm will evolve.

When these dynamic analysis algorithms are stored in each of the image management devices installed in each hospital, and analyzed by each image management device, it is difficult to reflect the daily evolution of the dynamic analysis algorithms on all image management devices. If a new dynamic analysis algorithm is developed, tile on-premises device is upgraded to accommodate the new algorithm by the version upgrade, which is not immediate. As a result, there will be differences in the type of analysis and analysis accuracy among image management devices and, consequently, among hospitals, resulting in a lack of fairness in medical services. From the viewpoint of providing simple, high-level medical services to many people, it is preferable from the viewpoint of homogeneity of medical services that the same analysis results be obtained regardless of which medical facility- the dynamic image was taken.

The present invention was made in view of the above-mentioned problems in the conventional technology, and is intended to provide highly accurate analysis results for the dynamic image while ensuring homogeneity.

To achieve at least one of the abovementioned objects, according to an aspect of the present invention, a recording medium reflecting one aspect of the present invention is a non-transitory recording medium storing a computer-readable program for modifying a dynamic analysis algorithm that performs dynamic analysis to a dynamic image obtained by performing dynamic imaging with radiation on a subject, the program causing a computer to perform: a process of receiving, from a first data collection device, a first data set that is an anonymized data set including a first dynamic image obtained by dynamic imaging wills radiation on a first subject and information obtained by a first test other than the dynamic imaging on the first subject; a process of receiving, from a second data collection device, a second data set that is an anonymized data set including a second dynamic image obtained by dynamic imaging with radiation on a second subject and information obtained by a second test other than the dynamic imaging on the second subject; and a process of modifying the dynamic analysis algorithm based on the first data set and the second data set.

To achieve at least one of the abovementioned objects, according to another aspect of the present invention, a dynamic analysis system reflecting one aspect of the present invention is a dynamic analysis system including: a dynamic analysis device that performs the program; the first data collection device; and the second data collection device.

To achieve at least one of the abovementioned objects, according to another aspect of the present invention, a dynamic analysis system reflecting one aspect of the present invention is a dynamic analysis system including: a dynamic analysis device that performs the program; and a hospital terminal that transmits the third dynamic image to the dynamic analysis device and receives the diagnosis support information from the dynamic analysis device.

To achieve at least one of the abovementioned objects, according to another aspect of the present invention, a dynamic analysis device reflecting one aspect of the present invention is a dynamic analysis device including: a storage in which a dynamic analysis algorithm that performs dynamic analysis to a dynamic image obtained by performing dynamic imaging with radiation on a subject is stored; a receiver that receives, front a first data collection device, a first data set that is an anonymized data set including a first dynamic image obtained by dynamic imaging with radiation on a first subject and information obtained by a first test other than the dynamic imaging on the first subject, and receives, from a second data collection device, a second data set that is an anonymized data set including a second dynamic image obtained by dynamic imaging with radiation on a second subject and information obtained by a second test other than the dynamic imaging on the second subject; and a hardware processor that modifies the dynamic analysis algorithm stored in the storage, based on the first data set and the second data set.

### BRIEF DESCRIPTION OF THE DRAWINGS

The advantages and features provided by one or more embodiments of the invention will become more fully understood from the detailed description given hereinbelow and the appended drawings which are given by way of illustration only, and thus are no intended as a definition of the limits of the present invention, wherein:

FIG. 1 is a system diagram of the dynamic analysis system in the embodiment of the present invention;

FIG. 2 is a block diagram showing the functional configuration of the dynamic analysis server;

FIG. 3 is a view showing an example of the data structure of the user management table;

FIG. 4 is the figure showing an example of the data structure of the data management table of the dynamic state data set database;

FIG. 5 is a block diagram showing the functional configuration of the hospital terminal;

FIG. 6 is a block diagram showing the functional configuration of the data collection server;

FIG. 7 is a block diagram showing the functional configuration of the service provider terminal;

FIG. 8 is a flowchart showing the in-hospital data collecting process performed by the data collection server;

FIG. 9 is a flowchart showing the data set transmission process performed by the data collection server;

FIG. 10 is a flowchart showing the data set reception process performed by the dynamic analysis server;

FIG. 11 is a flowchart showing the application learning process performed by the dynamic analysis server;

FIG. 12 is an illustration of supervised learning using a data set containing correct answer labels;

FIG. 13 is an illustration of unsupervised learning using a data set that does not contain the correct answer labels:

FIG. 14 is an illustration of the process of creating a normal model using normal case data;

FIG. 15 is an illustration of machine learning with different weights for each hospital that provided the data set;

FIG. 16 is a flowchart showing the application use permission determination process performed by the dynamic analysis server; and

FIG. 17 is a ladder chart showing the “process when using the dynamic analysis application”, which is performed by the dynamic analysis server and the hospital terminal.

### DETAILED DESCRIPTION

The embodiment of the dynamic analysis system of the present invention is described below with reference to the drawings. However, the scope of the invention is not limited to the disclosed embodiments or illustrated examples.

[Configuration of Dynamic Analysis System]

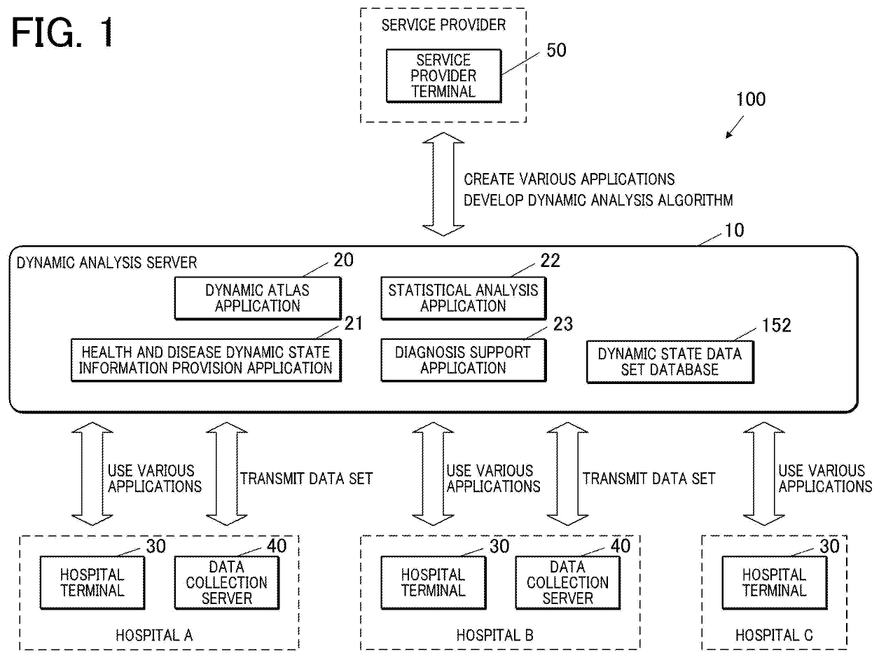

FIG. 1 shows the system configuration of the dynamic analysis system 100 in the embodiment of the present invention.

As shown in FIG. 1, the dynamic analysis system 100 includes: the dynamic analysis server 10; the hospital terminal 30 winch is used by the health care professionals belonging to each hospital; the data collection server 40 which manages medical information within each hospital; and the service provider terminal 50 used by the service provider administrator. The dynamic analysis server 10 can communicate data with the hospital terminal 30, the data collection server 40, and the service provider terminal 50 through a VPN (Virtual Private Network) connection, respectively.

The dynamic analysis server 10 is a dynamic analysis device that stores and manages the medical information uploaded from the data collection server 40 in the dynamic state data set database 152, in addition, the dynamic analysis server 10 provides various requested application software (hereinafter referred to as “applications”) and other services to the requesting hospital terminal 30 in response to a service use request from the hospital terminal 30. The applications provided by the dynamic analysis server 10 includes the dynamic atlas application 20, the health and disease dynamic state information provision application 21, the statistical analysis application 22, the diagnosis support application 23, etc.

The hospital terminal 30 is a computer device such as a PC (Personal Computer), tablet terminal, etc. used in each hospital. The hospital terminal 30 is used when displaying medical information stored in the dynamic analysis server 10 and using various applications provided by the dynamic analysis server M. The hospital terminal 30 accesses the dynamic analysis server 10 via a web browser and displays the processing results provided in the form of a web application. The hospital terminal 30 may also download a dedicated application and display the processing results using the dedicated application at the hospital terminal 30.

The data collection server 40 is a data collection device which collects data sets containing medical information such as the dynamic images obtained by performing dynamic imaging with radiation on patients in hospitals, information obtained by tests other than dynamic imaging on patients (including medical images, measurements, and other test results obtained by performing imaging other than dynamic imaging). The data collection server 40 performs anonymization processing (processing to make the information non-personally identifiable, such as anonymized information and pseudonymized information) on the patient information contained in the data set collected in the hospital, and uploads it to the dynamic analysis server 10. FIG. 1 shows that the data collection server 40 is located in each hospital, but the data collection server 40 can be located either inside or outside the hospital.

Only for hospitals that are designated to provide data sets to the dynamic analysis server 10, the data sets are provided to the dynamic analysis server 10 from the data collection server 40 of the hospital.

The service provider develops the dynamic analysis algorithm and creates various applications. The service provider terminal 50 installs the various applications created by the service provider to the dynamic analysis server 10. Not only a development company that develops an application using the dynamic state data set and provides this application on the dynamic analysis server 10, but also research institutions (universities, etc.) that conduct research and development of applications, and companies that develop and sell applications that incorporate dynamic analysis and various diagnosis support functions using API (Application Programming Interface) may be the service provider. There may be more than one service provider.

[Configuration of Dynamic Analysis Server]

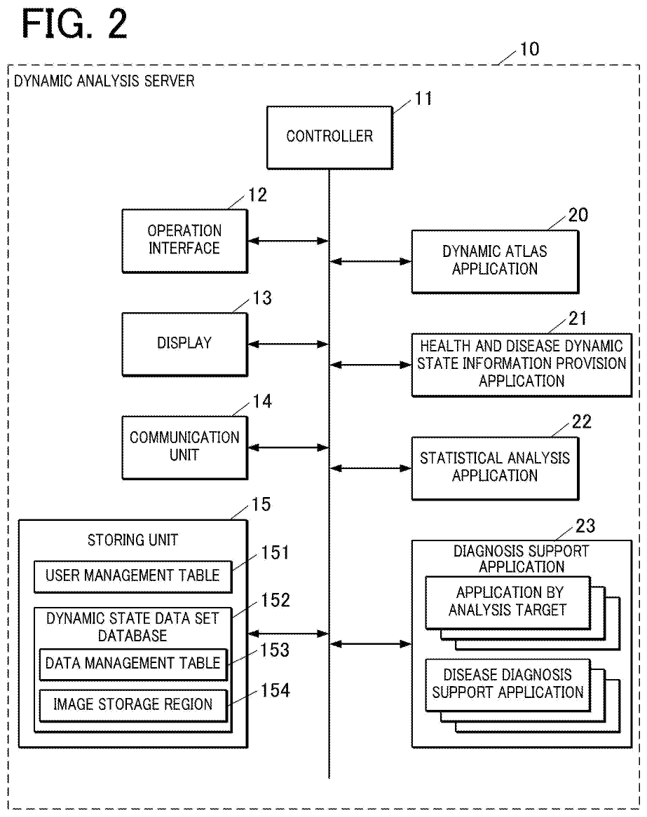

FIG. 2 shows the functional configuration of the dynamic analysis server 10.

As shown in FIG. 2, the dynamic analysis server 10 consists of the controller 11 (hardware processor), the operation interface 12, the display 13, the communication unit 14, the storing unit 15, the dynamic atlas application 20, the health and disease dynamic state information provision application 21, the statistical analysis application 22, the diagnosis support application 23, etc. The parts are connected by buses.

The controller 11, which consists of a CPU (Central Processing Unit), RAM (Random Access Memory), and the like, comprehensively controls the processing operations of each part of the dynamic analysis server 10. The CPU reads the system program and various processing programs stored in the storing unit 15, expands them in RAM, and performs various processes according to the expanded programs. The controller 11 may also be equipped with a GPU (Graphics Processing Unit) for AI processing.

The operation interface 12 includes a keyboard equipped with cursor keys, character/number input keys, and various function keys, and a pointing device such as a mouse. The operation interface 12 outputs instruction signals input by keyboard key operations and mouse operations to the controller 11.

The display 13 consists of a monitor such as a Liquid Crystal Display (LCD) and displays various screens according to the instructions of display signals input from the controller 11.

The communication unit 14 includes a network interface, etc., and transmits and receives data to and from external devices connected via a communication network.

The storing unit 15 includes HDD (Hard Disk Drive), SSD (Solid State Drive), etc., and stores various processing programs, parameters and files necessary for performing said programs. For example, the storing unit 15 stores the web server program to realize the function as a web server that communicates with a web browser installed in a hospital terminal 30 using the HTTP protocol to provide various web screens to the web browser, application programs that run on a web server, etc.

The storing unit 15 contains the dynamic atlas application 20, the health and disease dynamic state information provision application 21, the statistical analysis application 22 and the diagnosis support application 23, respectively.

The storing unit 15 stores the user management table 151 and the dynamic state data set database 152.

The user management table 151 is a table for managing information (user information) for each user (medical personnel) who uses the dynamic analysis system 100.

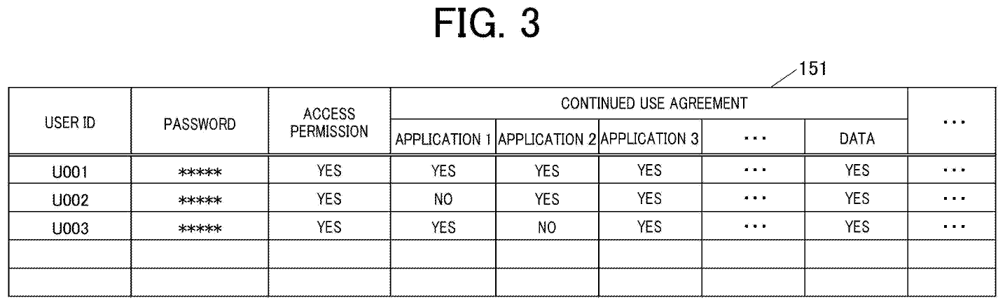

FIG. 3 shows an example of the data structure of the user management table 151.

In the user management table 151, the user ID, the password, the access permission, the continued use agreement, etc. are associated for each user.

The user ID is the user's identification information.

The password is used to authenticate the user when the user logs into the system.

The access permission is information indicating whether the user is permitted to access the dynamic analysis server 10 (indicated by “yes” in FIG. 3) or not (“no”).

The continued use agreement is information that indicates whether there is a contract (“yes” in FIG. 3) for the continued use of the services (each application and data) provided by the dynamic analysis server 10 or not (“no” in FIG. 3). The continued use agreement is determined for each application and data.

The dynamic state data set database 152 is a database that stores and manages data collected at each hospital and received from the data collection server 40 at each hospital. The dynamic state data set database 152 has the data management table 153 and the image storage region 154.

The data management table 153 is a table for managing the data sets collected at each hospital.

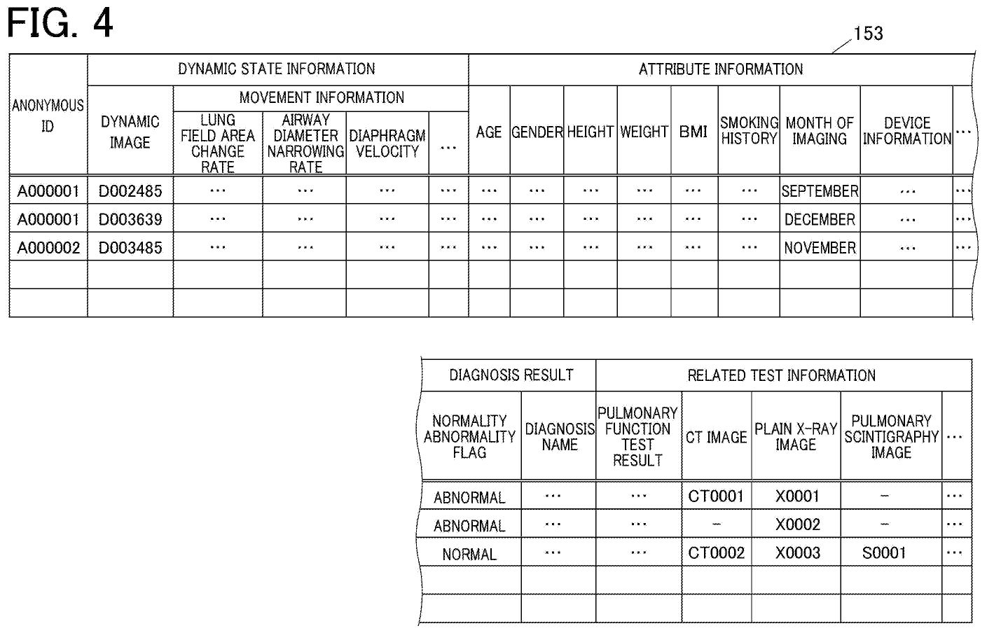

FIG. 4 shows an example of the data structure of the data management table 153.

The data management table 153 is a data management table in which the anonymous ID, the dynamic state information, the attribute information, the diagnosis result, and the related test information are stored so as to be associated with each other. In the data management table 153, one row of data is treated as one case data.

The anonymous ID is identification information to distinguish each subject (patient) that is the target of the dynamic imaging or the test other than dynamic imaging in a non-personally identifiable manner The anonymous ID is assigned a unique ID for each subject, but the anonymous ID cannot be used to identify the subject's corresponding patient ID, patient name, or other personal information.

The dynamic state information is the information obtained by performing dynamic imaging on the subject corresponding to the anonymous D. The dynamic state information includes the dynamic image and the movement information.

Dynamic imaging is the imaging of the dynamic state as the target, such as the morphological changes of tile expansion and contraction of the lungs associated with respirators, motion, the beating of the heart, and so on. in dynamic imaging, the subject is repeatedly irradiated with pulsed X-rays or other radiation at predetermined time intervals (pulsed irradiation) or continuously irradiated at low doses (continuous irradiation) to obtain multiple images that show the dynamic state of the subject. Dynamic imaging includes the moving image imaging, but does not include taking of still images while displaying the moving image (fluoroscopy).

The dynamic image is a series of images (image data) obtained by dynamic imaging. Each of the multiple images forming the dynamic image is called a frame image. The dynamic image includes the moving image, but does not include an image obtained by taking a still image while displaying the moving image. The dynamic image itself is stored in the image storage region 154 with tag information attached, and the tag information of the dynamic image is stored in the “dynamic image” field of the data management table 153.

The movement information is information that represents the movement of the subject (respiratory, cardiovascular, orthopedic, swallowing tissue, etc.) obtained by analyzing the dynamic image. The movement information includes the position obtained for each frame image, the velocity obtained from the difference between the frame images, and the maximum velocity and rate of change in size obtained analytically from such information.

The movement information includes, for example, quantified information on organizational dynamics such as lung field area change rate, airway diameter narrowing rate, and diaphragm velocity.

When the posterior ribs, sternum, clavicle, spine, diaphragm, and thorax are used as the imaging target part, the movement information includes time-series changes in position, time-series changes in velocity, maximum distance from the initial position, maximum and minimum velocities, etc.

When the heart is the imaging target part, the movement information includes the time-series change in size, time-series change in signal concentration, size change rate, signal concentration change rate, etc.

When the aortic arch is the imaging target part, the movement information includes the time-series change in signal value concentration, the rate of change in signal value concentration, etc.

When the airway is the imaging target part, the movement information includes the time-series changes in airway diameter size and airway diameter stenosis rate.

When the lung field is the imaging target part, the movement information includes the time-series change in lung field size, the rate of change of maximum and minimum lung field area, and the rate of change of signal value concentration.

The movement information may be information obtained from the data collection server 40 at each hospital, or information obtained by the dynamic analysis server 10 analyzing the dynamic image obtained from the data collection server 40.

The attribute information is information that indicates the attributes of the subject of the dynamic image that is the imaging target of the dynamic image or the attributes of the dynamic image. The attribute information includes, for example, age, gender, height, weight, BMI, smoking history, month of imaging, device information (information on the modality used for dynamic imaging), imaging conditions, imaging site, and imaging direction. The attribute information does not contain personally identifiable information (name, address, telephone number, etc.).

The diagnosis result is the diagnosis result for the dynamic image (the subject that is the imaging target of the dynamic image), and includes the normality abnormality flag, and the diagnosis name.

The normality abnormality flag indicates whether the subject contains the disease or not, and the normality abnormality flag is “normal” if the subject does not contain the disease, and “abnormal” if the subject contains the disease.

The diagnosis name is the name of the diagnosis (name of the disease, etc.) when the normality abnormality flag is “abnormal,” when the subject contains the disease. When the normality abnormality flag is “normal,” the diagnosis name is “no disease”. When the normality abnormality flag is “normal,” the diagnosis name may be left blank.

Diseases include, for example, diseases related to respiratory, cardiovascular, orthopedic, and swallowing. More specifically, COPD (chronic obstructive pulmonary disease) and interstitial pneumonia for respiratory diseases, heart failure and pulmonary embolism for cardiovascular diseases, and arthrosis and fracture for orthopedic diseases.

The normality abnormality flag is not only the normality abnormality flag for all diseases. The normality-abnormality flag for a specific disease may be added, for example, the normality abnormally flag for a respiratory disease or the normality abnormality flag for a cardiovascular disease.

In the data management table 153, records with the normality abnormality flag set to “normal” are normal cases, and records with the normality abnormality flag set to “abnormal” are abnormal cases.

The related test information is information obtained by performing tests other than dynamic imaging on the subject, medical images obtained by imaging other than dynamic imaging, measurements calculated from medical images obtained by imaging other than dynamic imaging, and test results (measured values, etc.) obtained by tests not involving imaging. Tests other than dynamic imaging include pulmonary function tests (e.g., spirometry), cardiac function tests (e.g., electrocardiography), scintigraphy test, CT scans, plant X-ray test, MRI tests, ultrasound test, and pulse oximeter test (SpO₂: transcutaneous arterial blood oxygen saturation). In FIG. 4, the related test information includes pulmonary function test results, CT images, plain X-ray images, and pulmonary scintigraphy images. Pulmonary function test results include volumetric capacity (VC), total lung capacity (TLC), functional residual capacity (FRC), residual volume (RV), RV/TLC, preliminary expiratory volume (ERV), volume in 1 second (FEV1), etc.

When the related test information includes the medical image obtained by imaging other than dynamic imaging (non-dynamic image), the medical image itself is stored in the image storage region 154 with tag information attached, and the tag information of the medical image is stored in the “CT image” field, “plain X-ray image” field, “pulmonary scintigraphy image” field, etc. in the data management table 153.

The image storage region 154 stores the medical images (the dynamic images, the non-dynamic images) uploaded from the data collection server 40 at each hospital.

The dynamic atlas application 20, the health and disease dynamic state information provision application 21, the statistical analysis application 22, the diagnosis support application 23 are applications provided to the hospital terminal 30, and perform processing in response to operations from the hospital terminal 30 and provides the processing results to the hospital terminal 30.

The dynamic atlas application 20 and the health and disease dynamic state information provision application glare content created using data from the dynamic state data set database 152, and are created by processing, process, sorting, etc. to the collected data. The dynamic atlas application 20 and the health and disease dynamic state information provision application 21 are also updated as data is added to the dynamic state data set database 152, along with the attribute information such as age group, gender, race, etc.

The dynamic atlas application 20 provides a normal case of the dynamic image, and provides the dynamic image and images obtained by existing modalities (CT/scintigraphy/MRI/ultrasound/still x-ray images, etc.) for comparison. For example, the dynamic atlas application 20 provides anonymized normal case data by site, gender, body shape, age, and device. The dynamic atlas application 20 displays the search results of normal cases that match the user-specified conditions (site, gender, body shape, age, imaging device, imaging facility, photographer, the diagnosis result, etc.) on the hospital terminal 30. For example, the user of the hospital terminal 30 uses the. dynamic atlas application 20 to check the typical dynamic images of the lungs in normal movement and the related test information (CT images, etc.) at that time.

The health and disease dynamic state information provision application 21 provides the dynamic images of the normal and abnormal cases (cases) per case and images obtained with existing modalities for comparison. For example, in the health and disease dynamic state information provision application 21, if the user at the hospital terminal 30 specifies “pulmonary embolism,” the user can view the typical dynamic images of the pulmonary embolism and the related test information (e.g., CT images) at the time of the embolism.

The statistical analysis application 22 and the diagnosis support application 23 are added as new applications when valuable analysis methods are developed. The statistical analysis application 22 and the diagnosis support application 23 are developed as correlations, tests, regression analysis, time-series data analysis, etc. are performed for each site and case.

The statistical analysis application 22 provides the results of statistical analysis of data used in tile dynamic atlas application 20 and the health and disease dynamic state information provision application 21. The statistical analysis application 22 provides statistical data (mean, variance, time-series data, etc.) by site, body shape, age, and device for normal and abnormal cases to the hospital terminal 30.

The diagnosis support application 23 is the dynamic analysis application that performs the dynamic analysis on the dynamic image for a given function or a given disease for the purpose of diagnosis support, and is realized by software processing in collaboration with the controller 11 and the dynamic analysis application program according to the content of the analysis.

The diagnosis support application 23 includes the application by analysis target and the disease diagnosis support application.

The application by analysis target performs the dynamic analysis (ventilation analysis, blood flow analysis, orthopedic analysis, diaphragm measurement, etc.) on the dynamic image for a given function. For example, the ventilation analysis application, blood flow analysis application, orthopedic analysis application, diaphragm measurement application, etc. are available as the application by analysis target.

The disease diagnosis support application performs the dynamic analysis on the dynamic image regarding the prescribed disease (COPD, interstitial pneumonia, etc.), determines whether the patient has the prescribed disease or not, and provides information used for such determination. For example, COPD diagnosis support application, interstitial pneumonia diagnosis support application, etc. are provided as disease diagnosis support applications.

When the dynamic analysis server 10 is accessed from the login account (the user ID and the password) corresponding to each user at the hospital terminal 30, the controller 11 determines whether or not to permit the user to use the application by referring to the user management table 151 in the storing unit 15. If the user ID and the password entered at the hospital terminal 30 is registered in the user management table 151, and the “access permission” corresponding to the entered user ID is “yes”(permitted) and the “continued use agreement” of the application corresponding to the entered user ID is “yes” (agreement is made), the controller 11 permits the user corresponding to this the user ID to use the application. If the user ID and the password entered at the hospital terminal 30 are registered in the user management table 151 and the “access permission” corresponding to the entered user ID is “yes”, even if the “continued use agreement” of the application corresponding to the entered user ID is “no”, the user corresponding to this user ID is permitted to use the application when the use agreement is newly made.

The controller 11 provides the requested medical information to the hospital terminal 30 from which the request is made in response to a viewing request from the hospital terminal 30.

The algorithm used for the program corresponding to each of the dynamic analysis applications (the application by analysis target, disease diagnosis support application) in the diagnosis support application 23 corresponds to the “dynamic analysis algorithm that performs dynamic analysis to a dynamic image obtained by performing dynamic imaging with radiation on a subject”. In other words, the storing unit 15, in which the program corresponding to each dynamic analysis application is stored, corresponds to the “storage in which the dynamic analysis algorithm is stored.

The dynamic analysis algorithm includes at least one of the algorithm by analysis target (e.g., ventilation analysis, blood flow analysis, orthopedic analysis, etc.) and disease diagnosis support algorithm (e.g., diagnosis support and differential diagnosis regarding COPD, interstitial pneumonia, etc.).

The communication unit 14 receives from the first data collection device (e.g., tile data collection server 40 at Hospital A) a first data set which is an anonymized data set containing the first dynamic image obtained by performing dynamic imaging with radiation on the first tile subject and information obtained by the first test other titan dynamic imaging on the first the subject. The communication unit 14 receives from the second data collection device (e.g., the data collection server 40 at Hospital B) a second data set which is an anonymized data set containing the second dynamic image obtained by performing dynamic imaging with radiation on the second subject and information obtained by the second test other than dynamic imaging on the second subject. In other words, the communication unit 14 functions as a receiver.

The “information obtained by tests other than dynamic imaging” includes numerical values (obtained by spirometry or other tests), images obtained by tests other than dynamic imaging (scintigraphy, CT scan, etc.), etc.

The “information obtained by tests other than dynamic imaging” may also include flags indicating normality or abnormality.

Here, the first and second tests may be different or the same. In addition, the first test in the first data set and second test in the second data set may be partially the same.

Each of the first and second tests includes at least one of pulmonary function tests (e.g., spirometry), cardiac function tests (e.g., electrocardiogram), scintigraphy, CT scan, plain X-ray test, MRI test, and-ultrasound test.

The first data set and the second data set may include correct answer labels and include teacher data used for machine learning.

The correct answer label includes at least one of a reading result for the dynamic image and the diagnosis result based on said reading result.

The reading result is information obtained by reading the dynamic image, including the position, size, type, etc. of the mass or other shadows.

The diagnosis result is the result of the diagnosis based on the reading result, and includes information such as tile distinction between normal/abnormal and the diagnosis name such as COPD.

The controller 11 stores the received first and second data sets in the dynamic state data set database 152 of the storing unit 15.

The correct answer label corresponds to the “diagnosis result” in the data management table 153 shown in FIG. 4.

The controller 11 modifies the dynamic analysis algorithm stored in the storing unit 15 (the storage) based on the first and second data sets. In other words, the controller 11 functions as a learning

In summary, the communication unit 14 receives data set from each of several data collection devices (such as the data collection servers 40). The data set is anonymized and includes the dynamic image obtained by dynamic imaging with radiation on a certain subject, and information obtained front tests other than dynamic imaging on the same subject as the subject targeted by the dynamic imaging.

The controller 11 performs machine learning for the dynamic analysis and diagnosis support based on the data sets received from each of the multiple data collection devices (data sets stored in the dynamic state data set database 152), and modifies the dynamic analysis algorithm.

The machine learning can use a Support Vector Machine (SVM), Random Forest, Deep Learning, etc.

The controller 11 inputs, via the communication unit 14, the third dynamic image obtained by performing dynamic imaging with radiation on the third subject (the diagnosis target patient).

The controller 11 outputs, via the communication unit 14, the diagnosis support information based on the dynamic analysis algorithm (learning model) modified by the controller 11 (the learning unit) for the third dynamic image. Specifically, the controller 11 inputs the third dynamic image received from the hospital terminal 30 to the dynamic analysis application selected front among the diagnosis support applications 23, obtains the diagnosis support information generated by the dynamic analysis application, and transmits the diagnosis support information to the hospital terminal 30 from which the third dynamic image was sent, via the communication unit 14.

The dynamic analysis application performs the dynamic analysis on the dynamic image to be diagnosed using Artificial Intelligence (AI). The dynamic analysis application generates the diagnosis support information using the dynamic analysis algorithm learned (modified) by machine learning based on the data set.

As for the dynamic analysis application which is learned based on the teacher data (data set containing the correct answer labels) among the diagnosis support applications 23, the information about the diagnosis of the dynamic image is output as the diagnosis support information. The controller 11 transmits (outputs) the information about the diagnosis of the dynamic image obtained from the dynamic analysis application via the communication unit 14 to the hospital terminal 30 which transmitted the third dynamic image. The information about the diagnosis of the dynamic image includes the diagnosis result (normal/abnormal, diagnosis name, etc.), reading result, etc.

As for the dynamic analysis application that has been learned with no teacher data (based on a data set that does not contain correct answer labels) among the diagnosis support applications 23, the classification information about the dynamic image is output as the diagnosis support information. The controller 11 transmits (outputs) the classification information about the dynamic image retrieved from the dynamic analysis application, via the communication unit 14, to the hospital terminal 30 which transmitted the third dynamic image. The classification information about the dynamic image is information indicating the classification (group) of the dynamic images into multiple groups by extracting data feature amounts through machine learning The person judges what kind of disease, symptom, etc. each group corresponds to (high possibility of COPD, low possibility of COPD, etc.).

Billing for the use of the application can be handled for each use, or on a flat-rate, pay-as-you-go, etc. basis.

For individual users, for example, users can reduce application purchase costs and initial installation costs by charging per application use.

Alternatively, a monthly or yearly usage contract may be concluded for individual users, and a fixed fee may be charged on a monthly or yearly basis. In this case, the user's initial installation cost can be reduced and the monthly or yearly payment cost can be clarified (subscription).

It is also possible to collect fees from individual users based on the amount of application usage (pay-as- you-go).

For the dynamic atlas application 20, it may be possible to allow users to use the dynamic image for free up to a predetermined number and to view the data without restrictions when a paid subscription is signed. Applications that do not charge (e.g., the dynamic atlas application 20, the health and disease dynamic state information provision application 21, etc.) may also be provided.

If the continued use agreement is not renewed or payment is not made by the user, the user loses the right to use the application.

The dynamic analysis server 10 may also provide AI applications (application software using machine learning and deep learning) such as the dynamic analysis and various diagnosis supports to researchers and developers of the dynamic analysis system using APIs. This allows researchers, development companies, etc. to create applications that incorporate the dynamic analysis and various diagnosis support functions provided in the form of APIs. When performing the application created in this way, the API on the dynamic analysis server 10 is accessed, and dynamic analysis and various diagnosis support functions are used as tools. In this case, the functions equivalent to the various applications provided by the API may be charged for each use, or may be charged on a pay-as-you-go basis. In addition, an annual contract may be made for each device that uses the functions equivalent to the various applications, and fees may be collected on an annual basis.

[Configuration of Hospital Terminal]

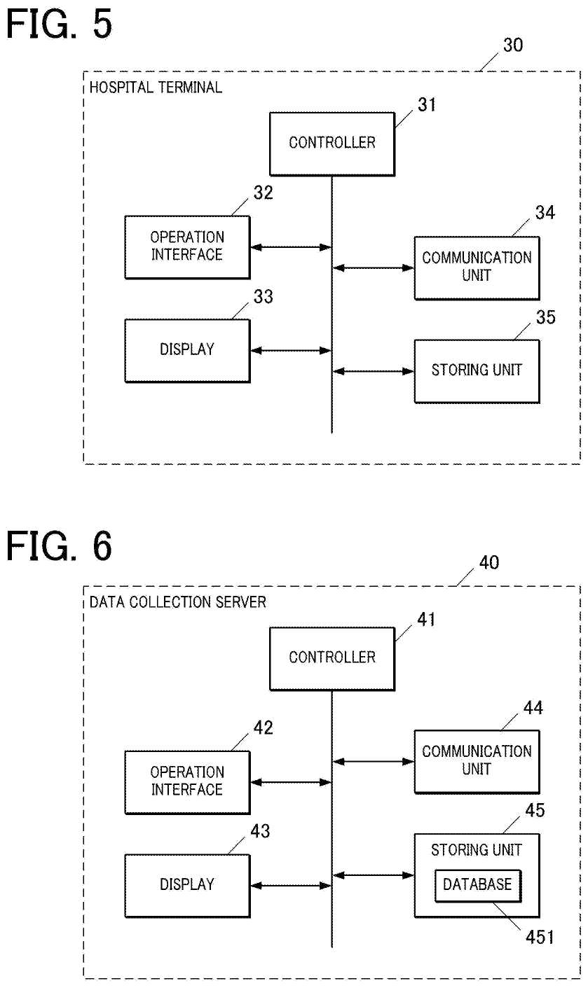

FIG. 5 shows the functional configuration of the hospital terminal 30.

As shown in FIG. 5, tile hospital terminal 30 includes the controller 31, the operation interface 32, the display 33, the communication unit 34, the storing unit 35, etc. The parts are connected by buses.

The controller 31 consists of a CPU. RAM, etc., and comprehensively controls the processing operations of each part of the hospital terminal 30. The CPU of the controller 31 reads the system program and various processing programs stored in the storing unit 35, expands them in RAM, and performs various processes according to the expanded programs.

The operation interface 32 includes a keyboard equipped with cursor keys, character/number input keys, and various function keys, and a pointing device such as a mouse, and outputs the instruction signals input by the key operation to the keyboard and the mouse operation to the controller 31. The operation interface 32 may also be equipped with a touch panel on the display screen of the display 33, in which case the instruction signals input via the touch panel are output to the controller 31.

The display 33 is composed of a monitor such as an LCD and displays various screens according to the instructions of the display signals input from the controller 31. For example, the display 33 displays various Web screens based on the data for displaying various Web screens received from the dynamic analysis server 10.

The communication unit 34 includes a network interface, etc., and transmits and receives data to and from external devices connected via a communication network.

The storing unit 35 includes HDD, SSD, etc., and stores various processing programs, parameters and files necessary for execution of said programs. For example, the storing unit 35 stores a web browser program to realize a web browser.

The controller 31 transmits, via the communication unit 34, the third dynamic image (tile dynamic image of the diagnosis target patient) obtained by performing dynamic imaging with radiation on the third subject to the dynamic analysis server 10 and receives the diagnosis support information from said dynamic analysis server 10.

The diagnosis support information is the output based on the dynamic analysis algorithm for the third dynamic

[Configuration of Data Collection Served]

FIG. 6 shows the functional configuration of the data collection server 40.

As shown in FIG. 6, the data collection server 40 includes the controller 41, the operation interface 42, the display 43, the communication unit 44, the storing unit 45, etc. The parts are connected by a bus.

Since the parts constituting the data collection server 40 are basically the same as those constituting the hospital terminal 30, only the parts characteristic to the data collection server 40 will be described, and the description will be omitted for the same configurations as those of the hospital terminal 30.

The storing unit 45 stores the database 451. The database 451 stores the dynamic image obtained from dynamic imaging performed in the hospital where the data collection server 40 is installed, information obtained by tests other than dynamic imaging. (including the non-dynamic images, test results such as measurements), etc.,

The data collection server 40 can be connected to an external HDD that can be accessed only by hospital personnel. The external HDD stores the anonymous ID correspondence table, in which information about patients in the hospital (patient It), patient name, address, phone number, etc.) is associated with the anonymous ID, in other words, in the external HDD, the anonymous ID correspondence table associates the patient (the subject) with the anonymous For patents for whom the anonymous ID has not, been generated, there is no record in the anonymous ID correspondence table. For security reasons, the anonymous ID correspondence table is not stored in the data collection server 40. The data collection server 40 does not allow access to the anonymous ID correspondence table on the external HDD for any purpose other than referencing and registering the anonymous ID of the target patient.

[Configuration of Service Provider Terminal]

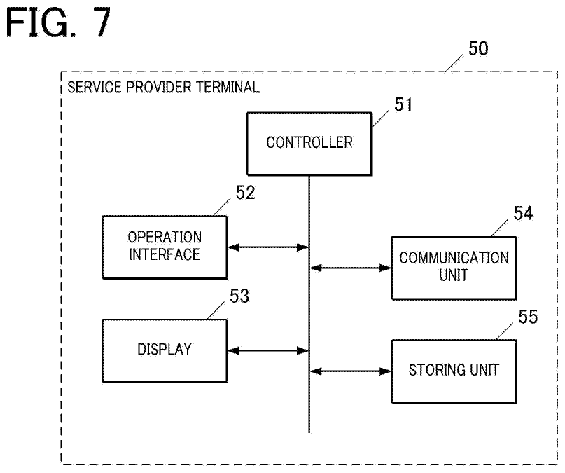

FIG. 7 shows the functional configuration of the service provider terminal 50.

As shown in FIG. 7, the service provider terminal 50 includes the controller 51, the operation interface 52, the display 53, the communication unit 54, the sliming unit 55, and so on. The parts are connected by buses.

The parts that make up the service provider terminal SC are basically the same as those that make up the hospital terminal 30, so the description is omitted.

[Operation of Dynamic Analysis System]

Next, the operation of the dynamic analysis system 100 is described.

<In-Hospital Data Collecting Process>

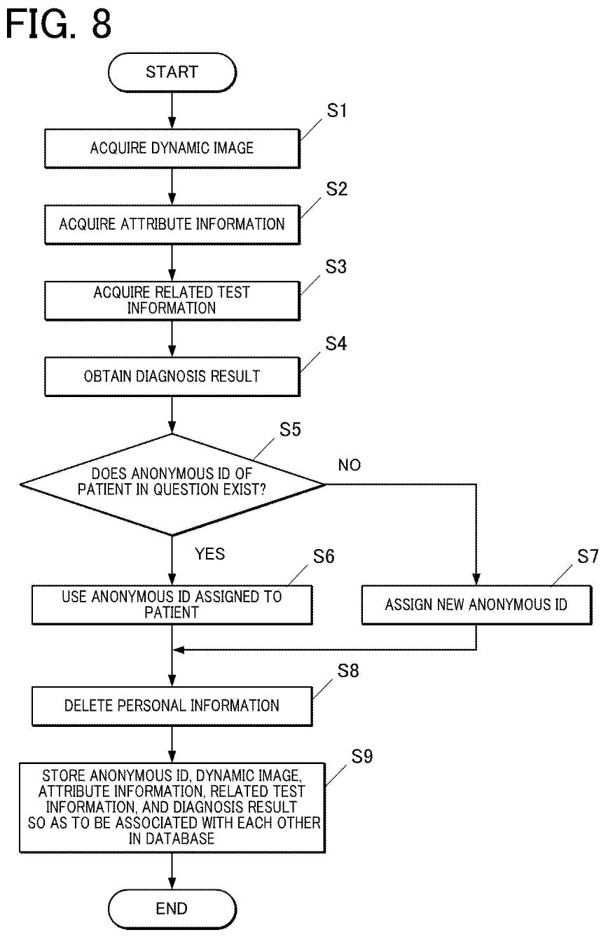

FIG. 8 is a flowchart showing the in-hospital data collecting process performed by the data collection server 40. The in-hospital data collecting process is realized by software processing through the cooperation of the CPU of the controller 41 and the program stored in the storing unit 45.

First, when dynamic imaging by radiation is performed on the subject with the dynamic imaging device in the hospital, the controller 41 of the data collection server 40 acquires the dynamic image from the dynamic imaging device via the communication unit 44 (step S1).

Next, the controller 41 obtains the attribute information of the subject determined as the imaging target in the dynamic imaging or the attribute information of the dynamic image (step S2). Specifically, the controller 41 acquires the attribute information from the supplementary information attached to the file of the dynamic image, or acquires the attribute information (patient information) corresponding to the subject from the electronic medical record device in the hospital via the communication unit 44.

The controller 41 may also display the attribute information input screen on the display 43 and accept input of the attribute information through the operation interface 42 by the medical personnel.

Next, the controller 41 acquires the related lest information pertaining to tests other than dynamic imaging performed on the subject that was determined as the imaging target in dynamic imaging (step S3). Specifically; the controller 41 extracts, from the test information stored in the storing unit 45, the test information of the same subject (patient) as that of the dynamic image acquired in step S1. Here, it is possible to limit the extraction to only the test information of tests performed within a predetermined period of time based on the date and time when the dynamic imaging was performed, or to only the test information of tests related to the imaging site of the dynamic imaging.

Next, the controller 41 obtains the diagnosis result for the dynamic image obtained in step S1. (step S4). Specifically, the controller 41 displays the diagnosis result input screen on the display 43 and accepts the input of normal (no disease)/abnormal (with disease) and diagnosis name (if disease exists) by the operation of the medical personnel from the operation interface 42. The diagnosis result may also include information such as annotations added to the dynamic image.

Next, the controller 41 determines whether or not the anonymous ID of the patient in question (the subject who is the imaging target in the dynamic imaging) exists (step S5). Specifically, the controller 41 accesses the external HDD via the communication unit 44, refers to the anonymous ID correspondence table stored in the external HDD, and determines whether Of not there is a record for the this patient.

If there is the anonymous ID for the patient in question (step S5; YES), the controller 41 uses the anonymous ID assigned to the patient (step S6).

if there is no anonymous ID for the patient in question in step S5 (step S5; NO), the controller 41 assigns a new unique anonymous ID for the patient in question (step S7). The controller 41 assigns the number irrelevant to the patient as the anonymous ID so that the patient is not identified from the anonymous ID. The controller 41 accesses the external HDD via the communication unit 44 and stores the patient information (patient ID, patient name, etc.) and the anonymous ID that was assigned in the anonymous ID correspondence table so as to be associated with each other.

After step S6 or step S7, the controller 41 deletes the personal information (patient ID, patient name, etc.) from each acquired information (step S8). This means that the data set that is later uploaded to the dynamic analysis server 10 is anonymized.

Next, the controller 41 associates the anonymous ID, the dynamic image, the attribute information, the related test information, and the diagnosis result, and stores them in the database 451 of the storing unit 45 (step S9).

This completes the in-hospital data collecting process.

In the in-hospital data collecting process, the dynamic image is acquired at the timing when dynamic imaging is performed in step S1. However, the timing of the acquisition of the dynamic image is not limited to this, and the dynamic image that has been taken and stored in advance may be acquired.

<Data Set Transmission Process>

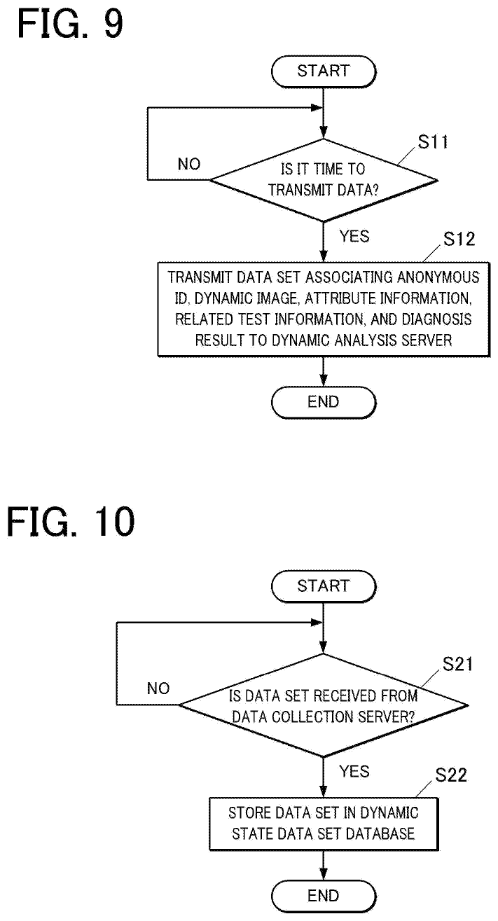

FIG. 9 is a flowchart showing the data set transmission process performed by the data collection server 40. The data set transmission process is realized by, software processing through the cooperation of the CPU of the controller 41 and the program stored in the storing unit 45.

The controller 41 of the data collection server 40 determines whether or not it is time to transmit data, (step S11). For example, the controller 41 determines that it is time to transmit data when it is a predetermined time, such as at night when the communication volume is calm, or when the communication volume is below a predetermined value. The controller 41 may also determine that it is time to transmit data when the amount of dam. in the database 451 of the storing unit 45 that has not yet been sent exceeds a predetermined value.

If it is not a data transmission timing (step S11; NO), the process returns to step S11 and the process is repeated.

In step S11, if it is time to transmit data (step S11; YES), the controller 41 transmits the data set (anonymized) associating the anonymous ID, the dynamic image, the attribute information, the related test information and the diagnosis result stored in the database 451 of the storing unit 45, to the VPN-connected dynamic analysis server 10 via the communication unit 44 (step S12).

Here, the data set transmitted from the data collection server 40 to the dynamic analysis server 10 may the movement information corresponding to the dynamic image.

This completes the data set transmission process.

After the data set transmission process, the controller 41 of the data collection server 40 may decide to delete the transmitted data set front the database 451. Alternatively, the controller 41 may leave the data set in the database 451 with the addition that it has been transmitted.

Alternatively, the data collection server 40 may transmit the data set to the dynamic analysis server 10 only if the user chooses to transmit the data set, according to the user's settings,

Since some hospitals have restrictions on communication with external networks, the data set may be stored on recording media or other media and physically handed over to the administrator of the dynamic analysis server 10, and the administrator may then import the data set from the recording media into the dynamic analysis server 10.

<Data Set Reception Process>

FIG. 10 is a flowchart showing the data set reception process performed by the dynamic analysis server 10. The data set reception process is realized by software processing through the cooperation of the CPU of the controller 11 and the program stored in the storing unit 15.

The controller 11 of the dynamic analysis server 10 determines whether or not it has received a data set (anonymized) from one of the data collection servers 40 connected to the VPN via the communication unit 14 (step S21).

If no data set is received from any of the data collection servers 40 (step S21; NO), the process returns to step S21 and is repeated.

If a data set is received from any of the data collection servers 40 in step S21 (step S21; YES), the controller 11 stores the received data set in the dynamic state data set database 152 of the storing unit 15 (step S22). Specifically, the controller 11 stores the information of the received data set so as to be associated in the data management table 153 (see FIG. 4) and stores the medical image (the dynamic image, the non-dynamic image) in the image storage region 154.

This completes the data set reception process.

By repeating the data set reception process, the dynamic analysis server 10 receives the anonymized data set associating the anonymous ID, the dynamic image, the attribute information, the related test information, and the diagnosis result from each of the data collection servers 40 of multiple hospitals. The related test information included in the data set can vary from data set to data set. For example, the communication unit 14 of the dynamic analysis server 10 receives a data set containing the dynamic images and pulmonary function test results from the data collection server 40 at Hospital A, and a data set containing the dynamic image and the CT image from the data collection server 40 of Hospital B.

The movement information stored in the data management table 153 of the dynamic state data set database 152 may be calculated by the dynamic analysis server 10 or included in the data set received from the data collection server 40.

The movement information includes the movement information related to respiratory, cardiovascular, orthopedic, swallowing, etc., depending on the corresponding dynamic image. The movement information related to the respiratory organs includes the lung field area change rate, airway diameter narrowing rate, diaphragmatic velocity, etc. The movement information related to the cardiovascular system includes the rate at which the heart wall moves. The movement information related to orthopedics includes the trajectory of bending and stretching of joints such as knees and elbows (position change information) and the speed at which the joints are extended.

For example, the controller 11 detects the position (region) of the lung field from the dynamic image of the frontal view of the chest and calculates the lung field area for each frame image. The controller 11 then calculates the rate of change of the lung field area based on the movement of the lung fields in the series of the dynamic images.

The controller 11 detects the position of the airway from the dynamic images of the frontal chest and calculates the airway diameter for each frame image. The controller 11 then calculates the airway diameter narrowing rate from the airway motion in the dynamic image series.

The controller 11 also detects the position of the diaphragm in the dynamic image (multiple frame images) of the frontal chest and calculates the diaphragm velocity between frame images. The controller 11 also calculates the maximum diaphragm velocity from the movement of the diaphragm in a series of the dynamic images.

When the movement information is calculated by the dynamic analysis server 10 or the data collection server 40, the diagnosis support application 23 (dynamic analysis application) corresponding to the movement information may be used.

<Application Learning Process>

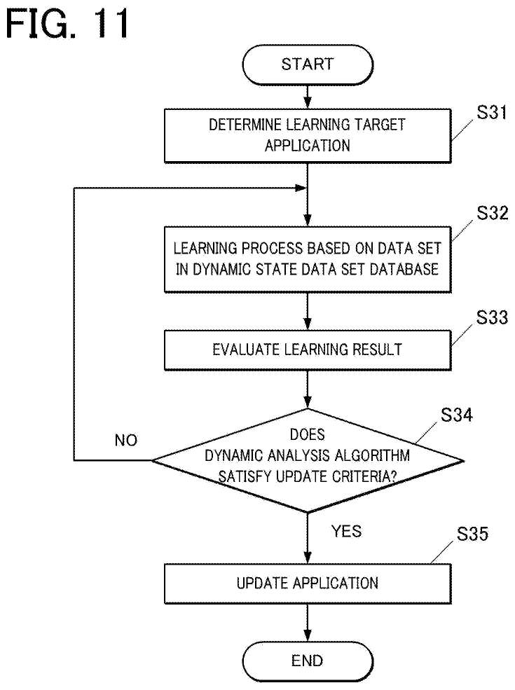

FIG. 11 is a flowchart showing the application learning process performed by the dynamic analysis server 10. The application learning process is realized by software processing through the cooperation of the CPU of the controller 11 and the programs stored in the storing unit 15.

First, the controller 11 of the dynamic analysis server 10 determines the dynamic analysis application to be the learning target (step S31). Specifically, the controller 11 chooses the application corresponding to the disease of the data set added to the dynamic state data set database 152 among the diagnosis support applications 23 as the dynamic analysis application to be the learning target. For example, if the diagnosis name in the data set is “COPD,” the dynamic analysis application for ventilation analysis is the learning target, and if the diagnosis name in the data set is “thrombus,” the dynamic analysis application for blood flow analysis is the learning target.

Next, the controller 11 reads the data sets stored in the dynamic state data set database 152 and, based on each data set, learns the dynamic analysis algorithm corresponding to the dynamic analysis application of the learning target (step S32). For the data sets stored in the dynamic state data set database 152, information indicating whether or not they were used for learning (modification) of each dynamic analysis application may be added so that only the data sets that have not been used for the dynamic analysis application of the learning target may be used for the current learning.

The dynamic analysis algorithm may have a function to perform image processing on the dynamic image, a function to perform the triage process, a function to perform the diagnosis judgment process, a function to performs the clustering process (sorting based on similarity) based on the features of the dynamic image, etc.

Triage generally refers to the prioritization of medical test and treatment according to the severity of the illness. The triage process in the dynamic analysis determines the priority of image confirmation for the dynamic image. For example, the triage process using clustering assigns priorities such as “abnormal/to be confirmed immediately (priority: high),” “likely abnormal (priority: medium),” “near normal (priority: low),” etc.

Next, the controller 11 evaluates the learning results of the dynamic analysis algorithm that has been learned (modified) (step S33). For example, the controller 11 evaluates whether or not the desired output results (analysis results) can be obtained from the sample data (the dynamic image) using the modified dynamic analysis algorithm.

Here, the controller 11 determines whether or not the learned dynamic analysis algorithm satisfies the predetermined update criteria (step S34). Specifically, the controller 11 automatically determines whether or not the update criteria are met based on the score for evaluating the learning results.

For the process of step S34, a person such as the person in charge of quality control of the dynamic analysis application may perform a final validity evaluation and update the application after a quality assurance.

If the learned dynamic analysis algorithm does not meet the update criteria (step S34; NO), the controller 11 returns to step S32 and performs the learning again, for example by excluding data that are, not suitable for learning.

In step S34, if the learned dynamic analysis algorithm satisfies the update criteria (step S34; YES), the controller 11 reflects the learned the dynamic analysis algorithm and updates the learning target application in the dynamic analysis server 10 (step S35).

This concludes the application learning process.

(Learning Example 1)

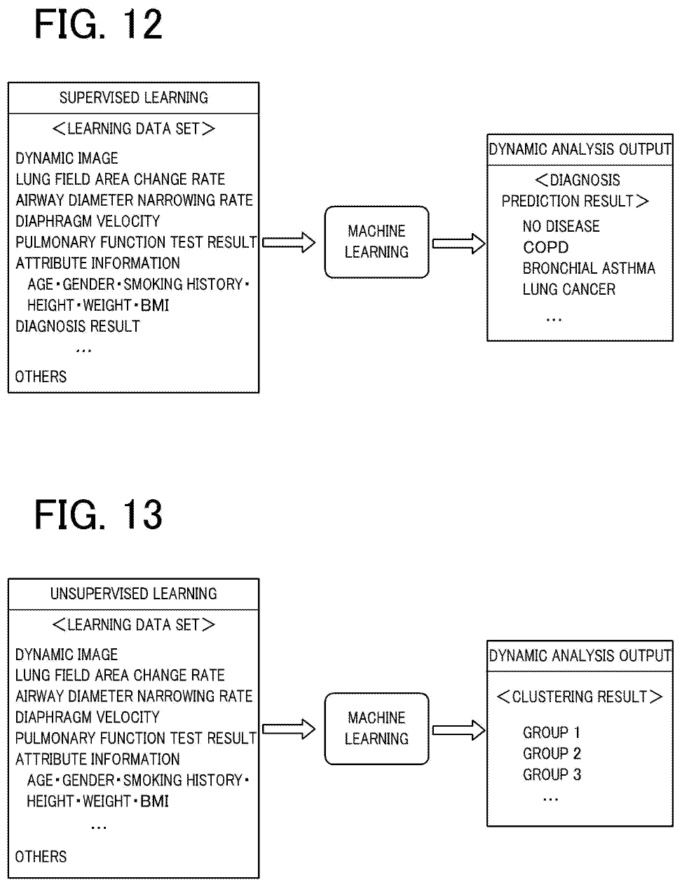

FIG. 12 is an illustration of supervised learning using a data set containing the correct answer label (diagnosis result). The controller 11 of the dynamic analysis server 10 generates the discriminator with the dynamic image, lung field area change rate, airway diameter stenosis rate, diaphragmatic velocity, pulmonary function test results, the attribute information (age, gender; smoking history, height, weight, BMA), etc. as input and with the diagnosis result (no disease, COPD, bronchial asthma, lung cancer, etc.) as output for each case data accumulated in the dynamic state data set database 152.

When using the dynamic analysis application from the hospital terminal 30, the dynamic analysis application uses the dynamic analysis algorithm including the learned discriminators to output diagnosis prediction results for the dynamic image to be the diagnosis result. Specifically, the dynamic analysis application calculates the movement information (lung field area change rate, airway diameter stenosis rate, diaphragmatic velocity, etc.) from the dynamic image that is the diagnosis target, and inputs the dynamic image, lung field area change rate, airway diameter narrowing rate, diaphragmatic velocity, pulmonary function test results, the attribute information (age, gender, smoking history, height, weight, BMI), etc. to the learned discriminator to obtain output results (diagnosis prediction results). However, as for the related test information and the attribute information, such as pulmonary function test results, only those which were obtained as information corresponding to the dynamic image as the diagnosis target may be used.

(Learning Example 2)

FIG. 13 is an illustration of unsupervised learning using a data set that does not contain correct answer labels. The controller 11 of the dynamic analysis server 10 performs machine learning by using the dynamic images, lung field area change rate, airway diameter narrowing rate, diaphragmatic velocity, pulmonary function test results, the attribute information (age, gender, smoking history, height, weight, BMI), etc., for each case data stored in the dynamic state data set database 152, and divides the dynamic image into multiple groups based on the similarity between the data (clustering).

When using the dynamic analysis application from the hospital terminal 30, the dynamic analysis application uses the dynamic analysis algorithm for grouping obtained by machine learning and outputs information (clustering results) indicating the groups to which the dynamic image as the diagnosis target belong. Specifically, the dynamic analysis application calculates the movement information (lung field area change rate, airway diameter stenosis rate, diaphragmatic velocity, etc.) from the dynamic images as the diagnosis target, and obtains the output result (clustering result) based on the dynamic image, lung field area change rate, airway diameter narrowing rate, diaphragmatic velocity, pulmonary function test results, the attribute information (age, gender, smoking history, height, weight BMI), etc. However, as for the related test information and the attribute information, such as pulmonary function test results, only those which were obtained as information corresponding to the dynamic image that is used as the diagnosis target run be used.

(Learning Example 3)

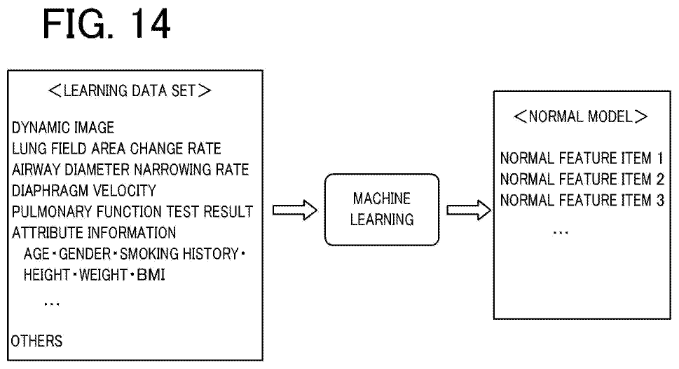

FIG. 14 is an illustration of the process of creating a normal model using normal case data. The controller 11 of the dynamic analysis server 10 constructs the normal model by machine teaming (support vector machines, random forests, deep learning, etc.) based on the dynamic image obtained by performing dynamic imaging on the subject that does not contain the disease, lung field area change rate, airway diameter narrowing rate, diaphragm velocity, pulmonary function test results, the attribute information (age, gender, smoking history, height, weight, BMI), and so on. The controller 11 automatically derives the normal feature items that characterize the “normal model”. For example, the controller 11 searches for the item that shows the relationship between height and BMI as normal feature item 1, item that shows the relationship between smoking history and pulmonary function test results as normal feature item 2, and item that shows the relationship between airway diameter stenosis rate and pulmonary function test results as normal feature item 3.

When using the dynamic analysis application from the hospital terminal 30, the dynamic analysis application determines whether the dynamic image that is the diagnosis target is normal or abnormal by calculating the degree of deviation from the normal model. The dynamic analysis application calculates the degree of deviation from the “normal model” of the information corresponding to the dynamic image that is the diagnosis target for each of the normal feature items 1, 2, 3, . . . . If any of the degrees of deviation corresponding to the respective normal feature items is larger than the predetermined threshold, it may be judged as abnormal, or the overall deviation degree may be recalculated by comprehensively judging each normal feature item, and if the overall deviation degree is larger than the predetermined threshold, it may be judged as abnormal There is no particular limitation on the method of judging whether or not the condition is normal or abnormal.

(Learning Example 4)

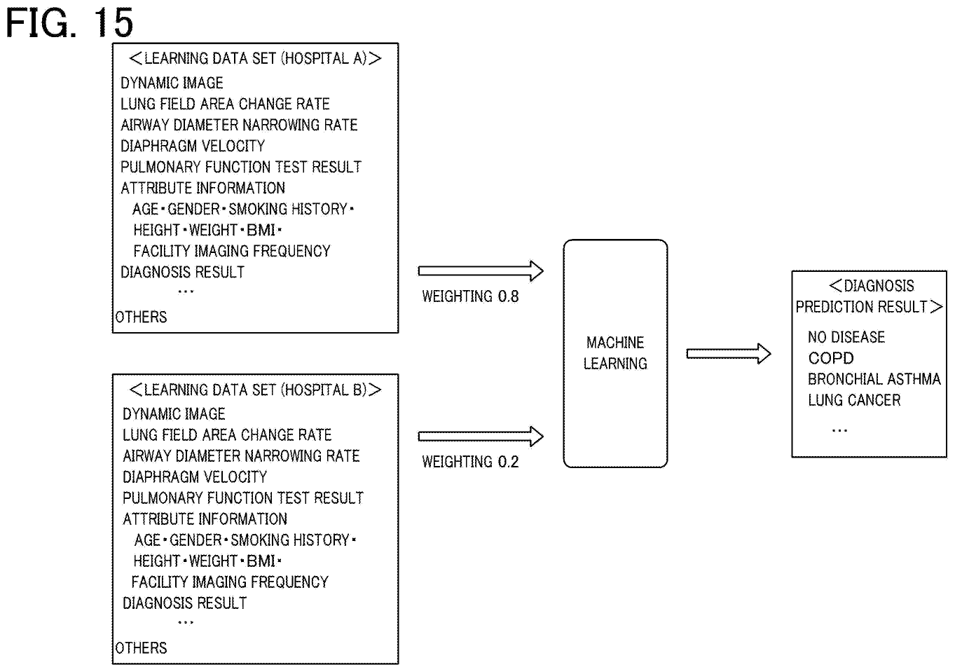

FIG. 15 is an image diagram of machine learning with the weighting-changed for each hospital that provided the data set. By using “facility imaging frequency” as the attribute information contained in the data set, the weighting of the data set of Hospital A, which has a relatively high frequency of imaging, is given higher weight than that of Hospital B (for example, the weighting of Hospital A is 0.8 and that of Hospital B is 0.2.), increasing the degree of reflection in the learning results. On the other hand, the data set of Hospital B which has a relatively low frequency of imaging is judged as not being accustomed to taking pictures and having little diagnostic experience, lowering the degree of reflection in the learning results. The learning example 4 is similar to the learning example 1 in that the dynamic image, lung field area change rate, airway diameter narrowing rate, diaphragm velocity, pulmonary function test results, the attribute information (age, gender, smoking history, height, weight, BMI), etc. are used as inputs and the diagnosis result (no disease, COPD, bronchial asthma, lung cancer, etc.) is used as output to generate the discriminator,

The case in which the weighting for machine learning is changed according to the “facility i imaging frequency” is described here. However, the data used for machine learning and deep learning may be weighted according to the imaging level of each photographer (number of images taken (experience), whether or not re-imaging is performed, required imaging time for each site, variance value of the validity (ROI misalignment) of images taken, etc.) and the presence of a medical specialist within the hospital.

The learning of the dynamic analysis algorithm may be performed outside of the dynamic analysis server 10. For example, the service provider may obtain a data set for learning from the dynamic analysis server 10, learn and evaluate the dynamic analysis algorithm, and then reflect the modified dynamic analysis algorithm to update the dynamic analysis application on the dynamic analysis server 10.

<Dynamic Atlas Update Method>

Next, the method of updating the dynamic atlas application 20 is described.

First, the administrator of the service provider uses the service provider terminal 50 to retrieve data from the dynamic analysis server 10, which is connected via VPN. The controller 51 of the service provider terminal 50 transmits a data acquisition request for the dynamic state data set database 152 to the dynamic analysis server 10 via the communication unit 54 and acquires the data set from the dynamic analysis server 10.

Next, a medical specialist (a physician with authority) selects data suitable for the dynamic atlas application 20 from the data set obtained from the dynamic analysis server 10. The medical specialist uses the selected data for clinical research, academic research, application development, etc.

Next, the medical specialist determines whether or not to update the dynamic atlas application 20, whether or not the selected data are data that should be added to the dynamic atlas application 20. Instead of the determination by the medical specialist, the dynamic atlas application 20 may be updated with standard (low deflection) image among the dynamic image with the normality abnormality flag indicating “normal”.

If the medical specialist decides not to update the dynamic atlas application 20, the data set is again obtained from the dynamic analysis server 10 and the data is selected.

If the medical specialist decides to update the dynamic atlas application 20, the administrator of the service provider performs update work of the dynamic atlas application 20 from the operation interface 52 of the service provider terminal 50 by the instruction from the medical specialist. The controller 51 of the service provider terminal 50 transmits the update instruction and update contents of the dynamic atlas application 20 to the dynamic analysis server 10 via the communication unit 54.

At the dynamic analysis server 10, the controller 11 updates the dynamic atlas application 20 based on the information received from the service provider terminal 50.

The health and disease dynamic state information provision application 21 is also updated under the supervision of a medical specialist, as is the dynamic atlas application 20.

<Application Use Permission Determination Process>

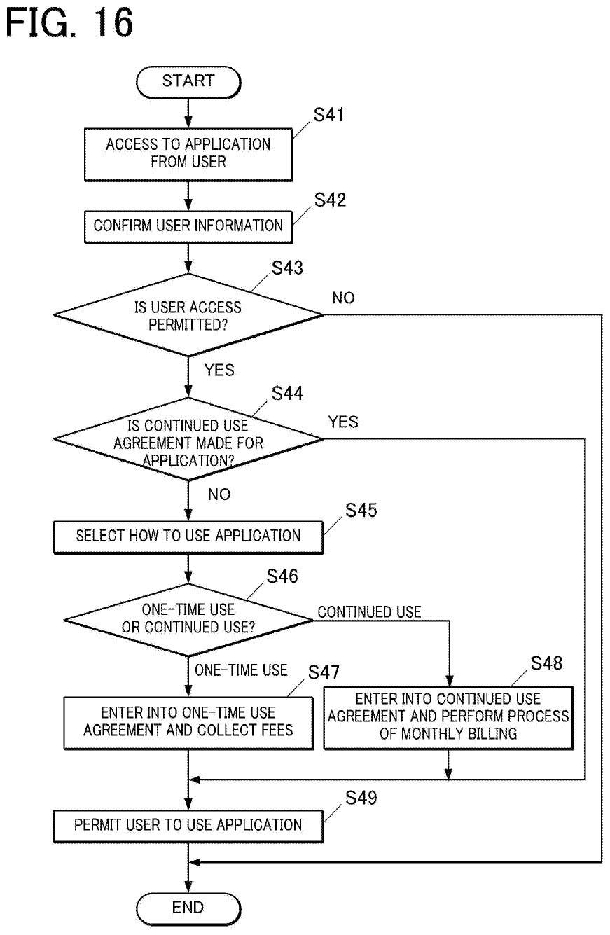

FIG. 16 is a flowchart showing the application use permission determination process performed by the dynamic analysis server 10. The application use permission determination process is realized by a software process in collaboration with the CPU of the controller 11 and the program stored in the storing unit 15.

First, at the hospital terminal 30, the user (medical personnel) operates the operation interface 32 and selects one of the applications provided by the dynamic analysis server 10, which is connected to the VPN, from the web browser. The controller 11 of the dynamic analysis server 10 accepts access to the selected application from the user via the communication unit 14 (step S41).

The controller 11 of the dynamic analysis server 10 transmits data for displaying the login screen to the hospital terminal 30 via the communication unit 14.

At the hospital terminal 30, when the user enters the user ID and the password by operating the operation interface 32 on the log-in screen displayed on the display 33, the controller 31 transmits the entered user ID and password to the dynamic analysis server 10 via the communication unit 34.

The controller 11 of the dynamic analysis server 10 obtains the user ID and the password entered from the operation interface 32 at the hospital terminal 30 via the communication unit 14. Then, the controller 11 checks the user information of the user corresponding to the user ID entered at the hospital terminal 30 by referring to the user management table 15 (see FIG. 3) stored in the storing unit 15 (step S42).

Next, the controller 11 determines whether or not the user access is permitted (step S43). Specifically, when the controller determines that a record corresponding to the combination of the user ID and the password entered from the hospital terminal 30 exists in the user management table 151 and “access permission” is “Yes”, the controller 11 determines that access is permitted.

If the user access is permitted (step S43; YES), the controller 11 determines whether the continued use agreement is made for the selected application (step S44). Specifically, the controller 11 refers to the user management table 151, and if the “continued use agreement” is “Yes” for the selected application in the record corresponding to the user ID entered from the hospital terminal 30, the controller 11 determines that the continued use agreement is made for the selected application.

If the continued use agreement is not made for the selected application (step S44, NO), the controller 11 allows the user of the hospital terminal 30 to select how to use the application (step S45). Specifically, the controller 11 displays a selection screen on the display 33 of the hospital terminal 30 to select one-time use or continued use, and accepts either selection on the operation interface 32.

If one-time use is selected by the user (step S46; one-time use), the controller 11 makes the user of the hospital terminal 30 enter into the one-time use agreement and collect fees (step S47). There is no limitation on the method of payment by the user.

If continued use is selected by the user in step S46 (step S46; continued use), the controller 11 makes the user of the hospital terminal 30 enter into the continued use agreement and performs the process of monthly billing (step S48). The controller 11 reflects in the user management table 151 that the continued use agreement has been made for the selected application. There is no limitation on the user's payment method. The billing method may be annual payment, pay-as-you-go billing, etc.

After step S47 or step S48, if the continued use agreement is made for the selected application in step S44 (step S44; YES), the controller 11 permits the user to use the application (step S49).

After step S49, or if the user access is not permitted in step S43 (step S43; NO), the application use permission determination process ends.

When the dynamic analysis server 10 permits the use of data in response to the access from the hospital terminal 30, the process is performed similarly to the application use permission determination process.

<Process When Using Dynamic Analysis Application>

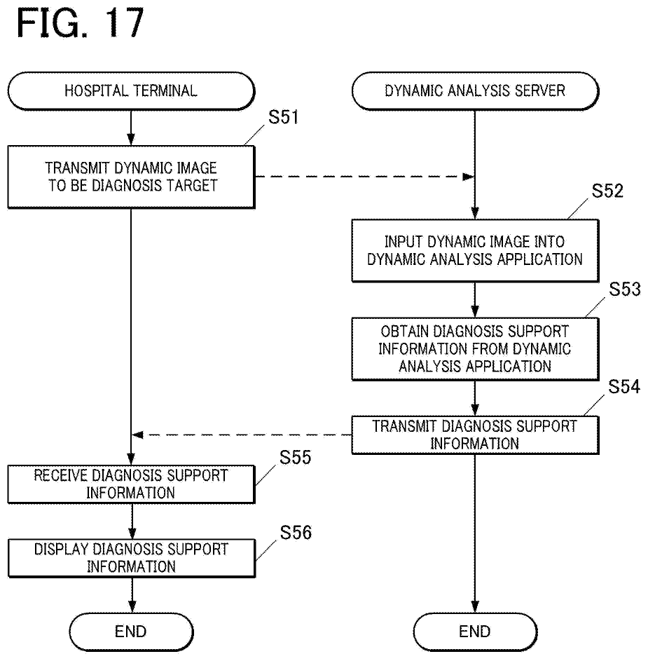

FIG. 17 is a ladder chart showing the “process when using the dynamic analysis application” performed by the dynamic analysis server 10 and the hospital terminal 30. The “process when using the dynamic analysis application” is the process that takes place after step S49, when one of the diagnosis support applications 23 (the dynamic analysis application) is accessed by the user in step S41 of the application use permission determination process. Data communication between the dynamic analysis server 10 and the hospital terminal 30 is performed via VPN.

At the hospital terminal 30, when the user (medical personnel) operates the operation interface. 32 to select the dynamic image (the third dynamic image) to be the diagnosis target, the controller 31 transmits the selected dynamic image to the dynamic analysis server 10 via the communication unit 34 (step S51). Here, the attribute information and the related test information corresponding to the dynamic image may be sent together with the dynamic image.

The controller 11 of the dynamic analysis server 10 receives the dynamic image transmitted from the hospital terminal 30 via the communication unit 14, and inputs this dynamic image to the dynamic analysis application selected by the user at the time of access (step S52). If the controller 11 receives the attribute information and the related test information along with the dynamic image, it inputs these data sets into the dynamic analysis application.

Next, the controller 11 obtains the diagnosis support information generated by the dynamic analysis from the dynamic analysis application (step S53). The diagnosis support information includes, for example, the analysis result report (including the diagnosis result, classification information, etc.), annotations on the dynamic image, measurement results such as distance measurements performed on the dynamic image, images to be referenced during diagnosis, etc.

Next, the controller 11 transmits the diagnosis support information to the hospital terminal 30 via the communication unit 14 (step S54).

The controller 31 of the hospital terminal 30 receives the diagnosis support information sent from the dynamic analysis server 10 via the communication unit 34 (step S55).

At the hospital terminal 30, the diagnosis support information is displayed on the display 33 (step S56). The user diagnoses the dynamic image while referring to the diagnosis support information.

This completes the “process when using the dynamic analysis application”.

In step S51, it is desirable to transmit the necessary data (uncompressed dynamic image data, compressed dynamic image data, still image data, etc.) according to the dynamic analysis application used, thereby reducing the amount of unnecessary data communication.

Although we have described the case where the dynamic analysis application (the dynamic analysis algorithm) selected by the user is used for the dynamic image, it is possible to perform multiple types of the dynamic analysis on the dynamic image at one time.

In addition, when using the functions corresponding to each application in API format; as in the “process when using the dynamic analysis application.”, the dynamic image (together with the attribute information, the related test information, etc. corresponding to the dynamic image, if necessary) is transmitted from the hospital terminal 30 to the dynamic analysis server 10, and the hospital terminal 30 receives the diagnosis support information from the dynamic analysis server 10.

As explained above, according to the embodiment, the dynamic analysis server 10 modifies (learns) the dynamic analysis algorithm based on the data sets collected at multiple data collection servers 40. This allows the dynamic analysis server 10 to provide highly accurate analysis results while ensuring homogeneity for the dynamic image, which is more important to analyze than a still image. Therefore, the dynamic analysis server 10 can provide the user with more reliable dynamic analysis results (such as the judgment result of whether the dynamic image is normal or abnormal, the diagnosis name that can be read from the dynamic image, etc.) to support diagnosis.

In particular, for the diagnosis support application 23, it is easy to increase the types of the analysis targets and improve the accuracy of the analysis by performing machine learning and deep learning using a large amount of data that is updated daily.

In addition, the dynamic analysis server 10 can provide new applications and data as needed while collecting data. By providing various applications on the dynamic analysis server 10, it is possible to centrally manage tile various applications and provide homogeneous information to the users at each hospital with the latest applications.

In addition, even after the dynamic analysis server 10 starts to be used in the dynamic analysis system 100, the accuracy of the dynamic analysis applications can be updated each time (post-market learning).

In addition, since the tests pertaining to the information contained in the data set can differ for each of the data collection server 40, the dynamic analysis algorithm can provide analysis results that have been learned also biking into account tests that are not performed at the hospital to which the user himself belongs.

In addition, by using information obtained from pulmonary function tests, cardiac function tests, scintigraphy tests, CT tests, plain X-ray tests, MRI tests, and ultrasound tests as information other than the dynamic images included in the data set, the dynamic analysis algorithm can be learned by considering what results were obtained in other tests for the subject that was tile target of the dynamic imaging.

Among tile dynamic analysis algorithms, the algorithm by analysis target can also perform ventilation analysis, blood flow analysis, orthopedic analysis, diaphragm measurement, etc. for the dynamic images.

In addition, among the dynamic analysis algorithms, the disease diagnosis support algorithm can determine whether or not a given disease (COPD, interstitial pneumonia, etc.) is present in the dynamic image, and. can provide information used for such determination.

In addition, the dynamic analysis application (the dynamic analysis algorithm), which is learned based on the data set containing the correct answer labels, can output information (normal or abnormal judgment result, diagnosis name, etc.) regarding the diagnosis of the dynamic image.

In addition, the dynamic analysis application (the dynamic state analysis algorithm), which is learned based on a data set that does not contain correct answer labels, can output classification information about the dynamic image as the diagnosis support information.

The description in the embodiment above is an example of the program, the dynamic analysis system and the dynamic analysis device pertaining to the present invention, and is not limited thereto. The detailed configuration and detailed operation of each device forming the system can also be changed as appropriate within the scope of the present invention.

For example, the users of the service provided by the dynamic analysis server 10 are not limited to medical personnel in hospitals, but may also include researchers belonging to research institutions such as universities, software developers, and the like.

In the embodiment above, the communication unit 14 (receiver) of the dynamic analysis server 10 receives the data sets sent from each data collection server 40 (data collection device) via VPN. However, it is also possible to receive the data sets from some of the data collection devices via VPN, and receive the data sets from other data collection devices by a method other than VPN.

Also, the communication unit 14 of the dynamic analysis server 10 may receive the data sets from each data collection device via, a dedicated line, Also, the communication unit 14 of the dynamic analysis server 10 may receive the data set from some data collection devices via a dedicated line and receive the data set from other data collection devices by a method other than the dedicated line.

Also, the communication unit 14 of the dynamic analysis server 10 may receive data sets from some data collection devices via a VPN and from other data collection devices via the dedicated line.

The functional units including the dynamic analysis server 10 may be divided into multiple devices. In other words, the dynamic analysis server 10 may consist of multiple devices. In such a case, the communication of data between the multiple devices, which is necessary to realize each function of the dynamic analysis server 10, is performed as appropriate. The dynamic analysis server 10 may also be constructed in the cloud and realized by a cloud server that may include multiple servers, storage, etc.

In the embodiment above, it is assumed that homogeneous analysis results are provided to each hospital, but it is also possible to provide each medical facility with an application that matches the device and conditions used at each facility, and to change the application provided according to the facility conditions (device information, trends in imaging data). Providing more suitable applications for each facility may increase the accuracy of analysis.

In addition, data obtained at other facilities may be also learned in the manner of transfer learning in order to improve the performance of the dynamic analysis.

In order to eliminate the influence of unintended changes to functions when performing machine learning, for example, when updating an application used at Hospital A, only data obtained by the same diagnosis device as at Hospital A and data with information on the diagnosis results of the medical specialist may be used for learning and updating the application. This can improve the learning effect in a way that removes the intrusion of data obtained. 30 by a different diagnosis device or data based on the diagnosis of a physician who is not familiar with the diagnosis.

In addition to providing various applications (the diagnosis support application 23, the dynamic atlas application 20, etc.) on the dynamic analysis server 10, for the applications frequently used at each hospital, it is also possible to download the various applications on the data collection server 40 and run the applications on the data collection server 40 from each of the hospital terminals 30. This method is also effective in case of connection failure to the dynamic analysis server 10.

In addition, at the hospital terminal 30, when the dynamic image of the diagnosis target is captured before selecting the application to be used, this dynamic image may be sent to the dynamic analysis server 10 so that the controller 11 of the dynamic analysis server 10 may have a recommendation function that suggests a process suitable for the dynamic image. For example, the controller it of the dynamic analysis server 10 may open the recommended diagnosis support application 23 on the hospital terminal 30, present the results of the process of the diagnosis support application 23 to the hospital terminal 30, suggest additional imaging orders to the hospital terminal 30, and the like.

The program for performing each process in each device may be stored in a portable recording medium. In addition, a carrier wave may be applied as a medium to provide program data via a communication line.

Although embodiments of the present invention have been described and illustrated in detail, the disclosed embodiments are made for purposes of illustration and example only and not limitation. The scope of the present invention should be interpreted by terms of the appended claims.

1. A non-transitory recording medium storing a computer-readable program for modifying a dynamic analysis algorithm that performs dynamic analysis to a dynamic image obtained by performing dynamic imaging with radiation on a subject, the program causing a computer to perform:
a process of receiving, from a first data collection device, a first data set that is an anonymized data set including a first dynamic image obtained by dynamic imaging with radiation on a first subject and information obtained by a first test other than the dynamic imaging on the first subject;
a process of receiving, from a second data collection device, a second data set that is an anonymized data set including a second dynamic image obtained by dynamic imaging with radiation on a second subject and information obtained by a second test other than the dynamic imaging on the second subject; and
a process of modifying the dynamic analysis algorithm based on the first data set and the second data set.

2. The recording medium according to claim 1, wherein the first test is different from the second test.

3. The recording medium according to claim 1, wherein
each of the first data set and the second data set includes a correct answer label, and
the correct answer label includes at least one of a reading result for the dynamic image and a diagnosis result based on the reading result.

4. The recording medium according to claim 1, wherein each of the first test and the second test includes at least one of a pulmonary function test, a cardiac function test, a scintigraphy test, a CT scan, a plain X-ray test, an MRI test, and an ultrasound test.

5. The recording medium according to claim 1, wherein the dynamic analysis algorithm includes at least one of an algorithm by analysis target and a disease diagnosis support algorithm.

6. The recording medium according to claim 1, wherein, in the processes of receiving the first data set and the second data set, at least one of the first data set and the second data set is received via a dedicated line.

7. The recording medium according to claim 1, wherein, in the processes of receiving the first data set and the second data set, at least one of the first data set and the second data set is received via, a virtual private network.

8. The recording medium according to claim 1, wherein, in the processes of receiving the first data set and the second data set, the first data set is received via a dedicated line and the second data set is received via a virtual private network.

9. The recording medium according to claim 1, wherein the program causes the computer to perform:
a process of inputting a third dynamic image obtained by performing dynamic imaging with radiation on a third subject; and
a process of outputting diagnosis support information based on the modified dynamic analysis algorithm, for the third dynamic image.

10. The recording medium according to claim 9, wherein
each of the first data set and the second data set includes a correct answer label.
the correct answer label includes at least one of a reading result for the dynamic image and a diagnosis result based on the reading result, and
in the process of outputting the diagnosis support information, information regarding diagnosis of the dynamic image is output as the diagnosis support information.

11. The recording medium according to claim 9, wherein, in the process of outputting the diagnosis support information, classification information regarding the dynamic image is output as the diagnosis support information.

12. A dynamic analysis system comprising:
a dynamic analysis device that performs the program according to claim 1;
the first data collection device: and
the second data collection device.

13. A dynamic analysis system comprising:
a dynamic analysis device that performs the program according to claim 9; and
a hospital terminal that transmits the third dynamic image to the dynamic analysis device and receives the diagnosis support information from the dynamic analysis device.

14. A dynamic analysis device comprising:
a storage in which a dynamic analysis algorithm that performs dynamic analysis to a dynamic image obtained by performing dynamic imaging with radiation on a subject is stored;
a receiver that
receives, from a first data collection device, a first data set that is an anonymized data set including a first dynamic image obtained by dynamic imaging with radiation on a first subject and information obtained by a first test other than the dynamic imaging on the first subject, and
receives, from a second data collection device, a second data set that is an anonymized data set including a second dynamic image obtained by dynamic imaging with radiation on a second subject and information obtained by a second test other than the dynamic imaging on the second subject; and
a hardware processor that modifies the dynamic analysis algorithm stored in the storage, based on the first data set and the second data set.
